(19) United States
(12) Patent Application Publication
(10) Pub. No.: US 2026/0102282 A1
(43) Pub. Date: Apr. 16, 2026

(54) EYE-PROCEDURE SURGICAL INSTRUMENT

(71) Applicant: THE CATHOLIC UNIVERSITY OF KOREA INDUSTRY-ACADEMIC COOPERATION FOUNDATION, Seoul (KR)

(72) Inventor: Dong Hyun JEE, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 19/296,705

(22) Filed: Aug. 11, 2025

(30) Foreign Application Priority Data
Oct. 16, 2024 (KR) 10-2024-0141559

(51) Int. Cl. A61F 9/007

(52) U.S. Cl. CPC A61F 9/007

(57) ABSTRACT

Disclosed is an eye-procedure surgical instrument including a handpiece which is grippable, a probe that protrudes from the handpiece and performs a procedure of a procedure area of an eye, a probe holder that supports one end of the probe and is accommodated in the handpiece, and a probe manipulating unit that moves the probe forward or rearward with respect to the procedure area.

### CROSS-REFERENCE TO RELATED APPLICATIONS

A claim for priority under 35 U.S. C. § 119 is made to Korean Patent Application No. 10-2024-0141559 filed on Oct. 16, 2024 in the Korean Intellectual Property Office, the entire contents of which are hereby incorporated by reference.

### BACKGROUND

Embodiments of the present disclosure described herein relate to an eye-procedure surgical instrument capable of finely moving a probe for an eye procedure.

As an example of an eye procedure, vitrectomy is a representative retinal surgery that removes a vitreous body in an eye.

When bleeding occurs, retinal detachment occurs, or a retinal proliferative membrane occurs inside the vitreous body, the bleeding should be removed, the retinal detachment should be re-attached, and the retinal proliferative membrane should be removed by performing the vitrectomy.

A case in which diabetic complications become severe in the eye is called proliferative diabetic retinopathy. In this case, the generated proliferative membrane is removed with a vitreous body cutter, and this vitreous body cutter actually plays the most important role in retinal surgery.

Meanwhile, when the vitrectomy is performed, the bleeding of the vitreous body is removed and the vitreous body attached to a retina is removed using the so-called vitreous body cutter in which a blade is formed at a distal end of a cylindrical cannula-shaped probe.

The vitreous body cutter has a hole at the distal end of the probe, the blade is attached to the hole, and the blade cuts the vitreous body while linearly reciprocating.

When the vitreous body cutter comes very close to the retina, complications resulting in cutting of a retinal tissue occur. As soon as the retinal tissue is touched during the procedure, retinal nerve cells may be damaged, and thus, blindness may be caused. When a retinal blood vessel is touched, the bleeding is caused, and the blindness is eventually caused, and when the vitreous body cutter comes into contact with the retina, the retina is cut off, and the retinal detachment is caused.

However, when the vitreous body cutter does not come close to the retina, the proliferative membrane or the like attached to the retina may not be removed, when the proliferative membrane is not removed, a decreased eyesight is caused, and when the vitreous body attached to the retina is left, the retinal detachment recurs, and thus re-operation should be required.

Thus, removing only the proliferative membrane attached to the retina without damaging the retina is one of the main processes of learning and training the retinal surgery.

However, even an experienced person may sometimes cause complications by damaging the retina while removing the proliferative membrane attached to the retina.

Currently, position of the vitreous body cutter, scissors for removing the proliferative membrane, etc. is adjusted to be close to the retina, and a height is adjusted by movement of a wrist and an entire hand.

However, the adjustment of the entire height using an eye-procedure surgical instrument, for example, the vitreous body cutter and the scissors, may not control accurate and precise movement.

A human body has a structure capable of much finer adjustment using a pressure of fingers than movement of the wrist.

Thus, adjusting of the height of the surgical instrument while raising and lowering the wrist and an arm is a rough operation, and the surgical instrument may not be finely adjusted.

On the other hand, a pressing pressure of the fingers, especially an index finger, may be adjusted incomparably fine.

Accordingly, the present applicant has developed an eye-procedure surgical instrument that may finely move a probe of the eye-procedure surgical instrument toward a procedure area using the pressure of the fingers, thus perform a precise procedure even by an unskilled person, and shorten a procedure time.

### SUMMARY

Embodiments of the present disclosure provide an eye-procedure surgical instrument capable of safely and precisely performing a procedure without damage to surrounding biological tissues, simply performing a procedure by an unskilled person, and also shortening a procedure time.

The aspects of the present disclosure are not limited to the aspects described above, and those skilled in the art will clearly understand other aspects not described from the following description.

According to an embodiment, an eye-procedure surgical instrument includes a handpiece which is grippable, a probe that protrudes from the handpiece and performs a procedure of a procedure area of an eye, a probe holder that supports one end of the probe and is accommodated in the handpiece, and a probe manipulating unit that moves the probe forward or rearward with respect to the procedure area, wherein the probe manipulating unit includes an elevation rod elevatably provided in the handpiece to cross a forward/rearward direction of the probe, a driving rack having a plurality of gear teeth formed at one end of the elevation rod in an elevation direction of the elevation rod, a pinion having a plurality of gear teeth engaged with the gear teeth of the driving rack and rotated while being engaged with the driving rack according to elevation of the elevation rod, and a driven rack of which a plurality of gear teeth engaged with the gear teeth of the pinion are formed at one end of the probe holder in the forward/rearward direction of the probe and which moves forward or rearward toward the procedure area by forward/rearward rotation of the pinion.

Here, the probe manipulating unit may include a push button that is provided at a free end of the elevation rod protruding from the handpiece and lowers the elevation rod and an elastic member that is provided between the push button and an outer surface of the handpiece and generates an elastic force to raise the elevation rod.

Further, the probe manipulating unit may further include a probe movement adjusting unit that controls lowering of the probe in stages and finely adjusts the forward movement of the probe toward the procedure area.

The probe movement adjusting unit may include a plurality of locking grooves recessed in the handpiece at intervals in the elevation direction of the elevation rod and a locking protrusion provided on an outer surface of the elevation rod and selectively locked by any one of the plurality of locking grooves.

The probe may be any one of a cutting cutter, forceps, scissors, and a retractor.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view of an eye-procedure surgical instrument according to an embodiment of the present disclosure;

FIG. 2 is a diagram of a probe manipulating unit of the eye-procedure surgical instrument according to the embodiment of the present disclosure;

FIG. 3 is a perspective view of an elevation rod of FIG. 2; and

FIGS. 4 and 5 are views illustrating processes of operating the eye-procedure surgical instrument according to the embodiment of the present disclosure.

### DETAILED DESCRIPTION

Advantages and features of the present disclosure and a method of achieving the advantages and the features will become apparent with reference to embodiments described below in detail together with the accompanying drawings. However, the present disclosure is not limited to embodiments disclosed below but may be implemented in various different forms, and the embodiments are provided only to make the disclosure of the present disclosure complete and to completely inform the scope of the present disclosure to those skilled in the art to which the present disclosure pertains.

Terms used in the specification are intended to describe embodiments and are not intended to limit the present disclosure. In the specification, a singular form also includes a plural form unless specifically mentioned in a phrase. The terms “comprises” and/or “comprising” used in the specification do not exclude the presence or addition of one or more other components other than the mentioned components. Throughout the specification, the same reference numerals refer to the same components, and the term “and/or” includes each and all combinations of one or more of components mentioned. Although “first,” “second,” and the like are used to describe various components, it is apparent that these components are not limited by these terms. These terms are only used to distinguish one component from another component. Thus, it is apparent that a first component mentioned below may be a second component within the technical spirit of the inventive concept.

Unless otherwise defined, all terms (including technical and scientific terms) used in the specification may be used as meanings commonly understood by those skilled in the art to which the present disclosure pertains. Further, terms defined in a commonly used dictionary are not interpreted ideally or excessively unless explicitly and specifically defined.

Hereinafter, the present disclosure will be described in detail with reference to the accompanying drawings.

Prior to description, in an eye-procedure surgical instrument in the present embodiment, it is described as an example that a probe is a cutting cutter for resecting a vitreous body of an eye, but the technical spirit according to the present disclosure reveals in advance that the probe of the eye-procedure surgical instrument may be any one of forceps, scissors, and retractors.

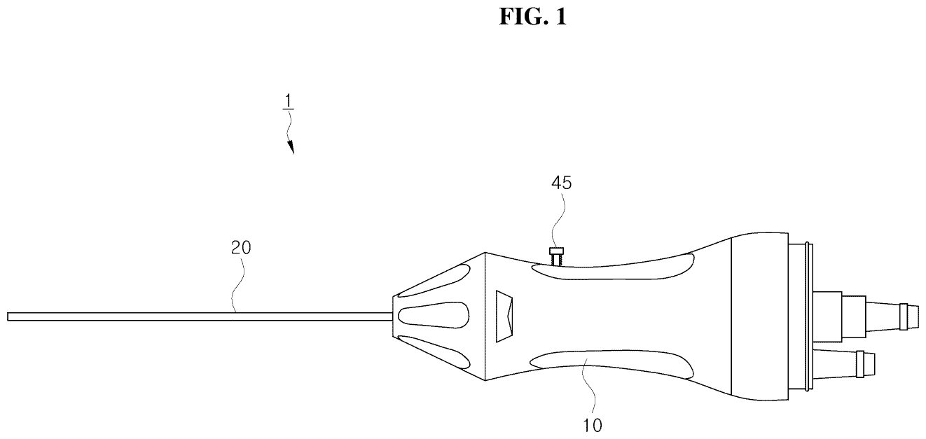

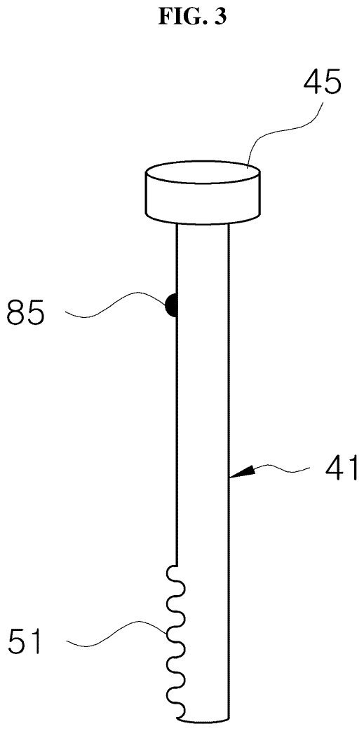

FIGS. 1 to 3 illustrate an eye-procedure surgical instrument according to an embodiment of the present disclosure.

As illustrated in these drawings, an eye-procedure surgical instrument 1 according to the embodiment of the present disclosure includes a handpiece 10, a probe 20, a probe holder 30, and a probe manipulating unit 40.

The handpiece 10 has a shape that may be easily gripped by an operator and forms a chamber 15 in which the probe holder 30 is accommodated. Further, a portion of the probe manipulating unit 40 is accommodated in the chamber 15.

The probe 20 has a predetermined length and protrudes from the handpiece 10.

A cutting blade 25 for cutting a procedure area of the eye, for example, the vitreous body of the eye, is formed at a free end of the probe 20. The cutting blade 25 is formed by partially cutting an outer circumference of the probe 20 at a predetermined width.

The probe 20 may move forward or rearward toward the procedure area of the eye by manipulating the probe manipulating unit 40, and accordingly, and the cutting blade 25 may cut the procedure area of the eye while moving forward or backward with respect to the procedure area.

The probe holder 30 has a bar shape having a certain length, supports one end of the probe 20, and is accommodated in the chamber 15 of the handpiece 10.

The probe manipulating unit 40 supports the one end of the probe 20 and moves forward or backward toward the procedure area of the eye by the manipulation.

The probe manipulating unit 40 moves the probe 20 and the probe holder 30 forward or backward toward the procedure area of the eye.

The probe manipulating unit 40 includes an elevation rod 41, a driving rack 51, a pinion 61, and a driven rack 71.

The elevation rod 41 has a bar shape having a certain length and is elevatably provided on the handpiece 10 to cross the forward and backward direction of the probe 20.

Meanwhile, a push button 45 for raising the elevation rod 41 easily using a finger of the operator is provided at a free end of the elevation rod 41 protruding from the handpiece 10.

The driving rack 51 is provided at an end opposite to the elevation rod 41 in which the push button 45 is provided.

In the driving rack 51, a plurality of gear teeth having a certain standard are formed at a certain length in an elevation direction of the elevation rod 41, e.g., a longitudinal direction of the elevation rod 41.

The pinion 61 has an external gear shape, has the same standard as the gear teeth of the driving rack 51 on an outer circumference of the pinion 61, and has a plurality of gear teeth rotated while being engaged with the gear teeth of the driving rack 51.

The pinion 61 is coupled to a rotary shaft 65 rotatably supported by the handpiece 10.

Accordingly, as the elevation rod 41 is raised and lowered, the pinion 61 rotates forward and backward about the rotary shaft 65.

The driven rack 71 is provided at an end opposite to the probe holder 30 on which the probe 20 is mounted.

In the driven rack 71, a plurality of gear teeth having the same standard as the gear teeth of the pinion 61 and rotated while being engaged with the gear teeth of the pinion 61 may be formed in a certain length in the forward and rearward direction of the probe 20, e.g., a longitudinal direction of the probe holder 30.

Accordingly, as the pinion 61 rotates forward and rearward, the probe 20 and the probe holder 30 linearly reciprocate while moving forward and backward toward the procedure area.

Further, the probe manipulating unit 40 may further include an elastic member 75.

The elastic member 75 is provided between the push button 45 and an the outer surface of the handpiece 10 and generates an elastic force to raise the elevation rod 41.

Accordingly, after the operator presses the push button 45 with his/her finger to lower the elevation rod 41, when the operator releases the finger from the push button 45, the elevation rod 41 is raised by the elastic force of the elastic member 75. In this way, by pressing or releasing the push button 45 with the finger, the operator may easily move the probe 20 forward or rearward toward the procedure area and perform the procedure safely and precisely.

Meanwhile, the eye-procedure surgical instrument 1 according to the embodiment of the present disclosure may further include a probe movement adjusting unit 80 that controls the lowering of the probe 20 in stages and finely controls the forward movement of the probe 20 toward the procedure area.

The probe movement adjusting unit 80 includes a plurality of locking grooves 81a, 81b, and 81c and a locking protrusion 85.

The plurality of locking grooves 81a, 81b, and 81c are recessed and formed in the handpiece 10 at intervals in an elevation direction of the elevation rod 41.

Hereinafter, for convenience of description, a locking groove positioned outside the handpiece 10 is referred to as the first locking groove 81a, a locking groove positioned in the middle of the handpiece 10 is referred to as the second locking groove 81b, and a locking groove positioned inside the handpiece 10 is referred to as the third locking groove 81c.

The plurality of locking grooves 81a, 81b, and 81c have a partial arc shape and are continuously formed in the longitudinal direction of the elevation rod 41. Here, in the present embodiment, it is illustrated that three locking grooves are provided, but the present disclosure is not limited thereto, and a plurality of locking grooves may be provided.

The locking protrusion 85 has a cross-sectional shape corresponding to the locking grooves 81a, 81b, and 81c, protrudes from an outer surface of the elevation rod 41, and is selectively locked by any one of the plurality of locking grooves 81a, 81b, and 81c.

Further, when the locking protrusion 85 is locked by the first locking groove 81a, the probe 20 of the eye-procedure surgical instrument 1 according to the embodiment of the present disclosure has a state of being positioned in an initial position before the procedure.

Accordingly, as the locking protrusion 85 is locked by any one of the plurality of locking grooves 81a, 81b, and 81c, the forward and rearward movement of the probe 20 with respect to the procedure area may be finely adjusted in stages.

Here, when the locking protrusion 85 is locked by the locking grooves 81a, 81b, and 81c, a “click” sound is generated, and accordingly, the operator may recognize the “click” sound during the procedure and thus easily know that the locking protrusion 85 is locked by the locking grooves 81a, 81b, and 81c.

With this configuration, a process of performing the procedure while finely moving the probe 20 toward the procedure area using the eye-procedure surgical instrument 1 according to the embodiment of the present disclosure will be described below.

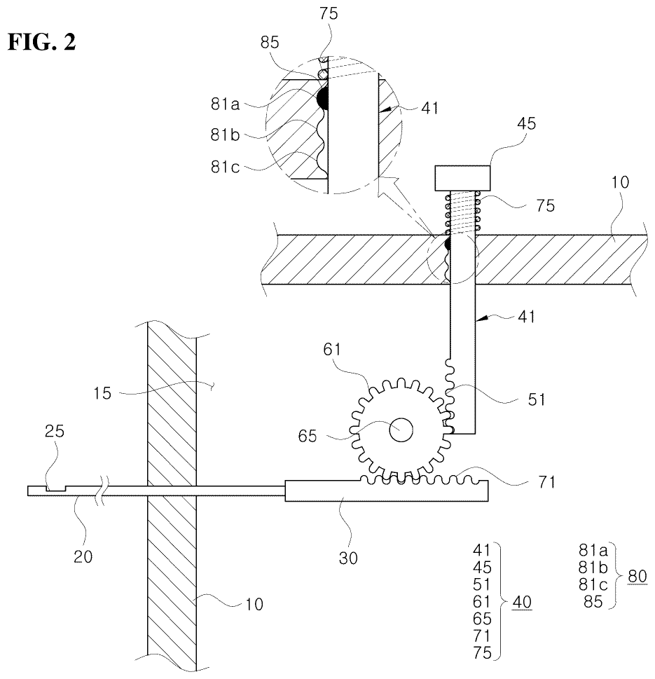

First, as illustrated in FIG. 2, in a state in which the locking protrusion 85 is locked by the first locking groove 81a, when the operator fixes the wrist and presses the push button 45 with his/her finger so that the locking protrusion 85 is locked by the second locking groove 81b, the elevation rod 41 is primarily lowered.

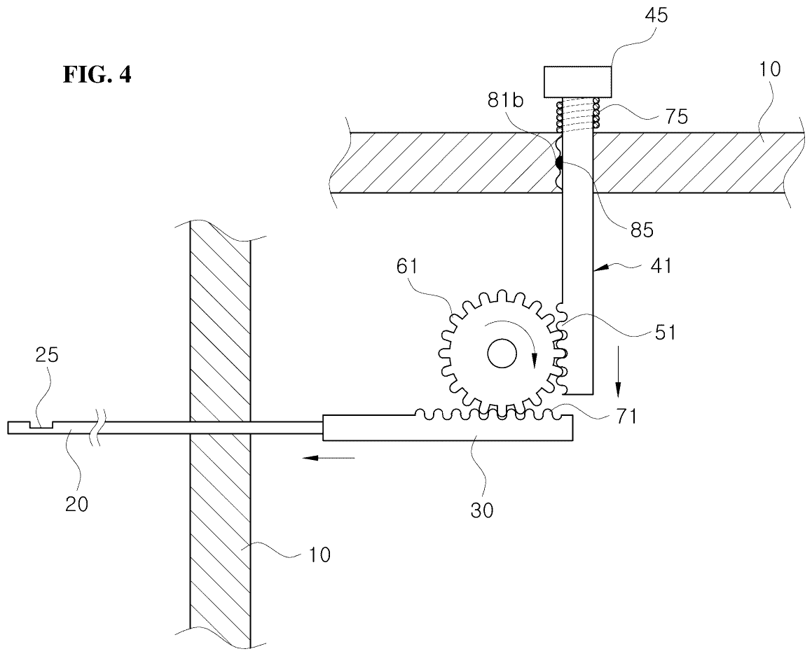

As the elevation rod 41 is primarily lowered, the driving rack 51 is also primarily lowered while the elastic member 75 is compressed, and as illustrated in FIG. 4, the driving rack 51 rotates the pinion 61 forward clockwise in correspondence to a distance by which the driving rack 51 is primarily lowered.

As the pinion 61 rotates forward, the driven rack 71 moves forward toward the procedure area by a primary movement distance corresponding to a distance by which the driving rack 51 is primarily lowered in a leftward direction of FIG. 4.

Thus, as the driven rack 71 moves forward by the primary movement distance, the probe holder 30 and probe 20 also move forward by the primary movement distance toward the procedure area.

Further, after the probe 20 moves forward by the primary movement distance toward the procedure area, when the operator releases the push button 45 by further slightly pressing the push button 45 while his/her wrist is fixed so that the locking protrusion 85 is separated from the second locking groove 81b, as illustrated in FIG. 2, by the elastic force generated while the elastic member 75 is tensioned, the elevation rod 41 is raised, the locking protrusion 85 is locked by the first locking groove 81a, the driving rack 51 is also raised as the elevation rod 41 is raised, and the pinion 61 rotates rearward counterclockwise. Further, as the pinion 61 rotates rearward, the driven rack 71 moves in a rightward direction of FIG. 4 by a movement distance corresponding to a distance by which the driving rack 51 is raised. Thus, the probe 20 and the probe holder 30 move rearward from the procedure area, and as illustrated in FIG. 2, the elevation rod 41 and the probe 20 are restored to their initial

### Positions.

Further, when the operator wants to move the probe 20 toward the procedure area by a movement distance greater than the primary movement distance, and when the operator presses the push button 45 with his/her finger while his/her wrist is fixed so that the locking protrusion 85 is locked by the second locking groove 81b, the elevation rod 41 is secondarily lowered by a distance greater than the primary lowering distance.

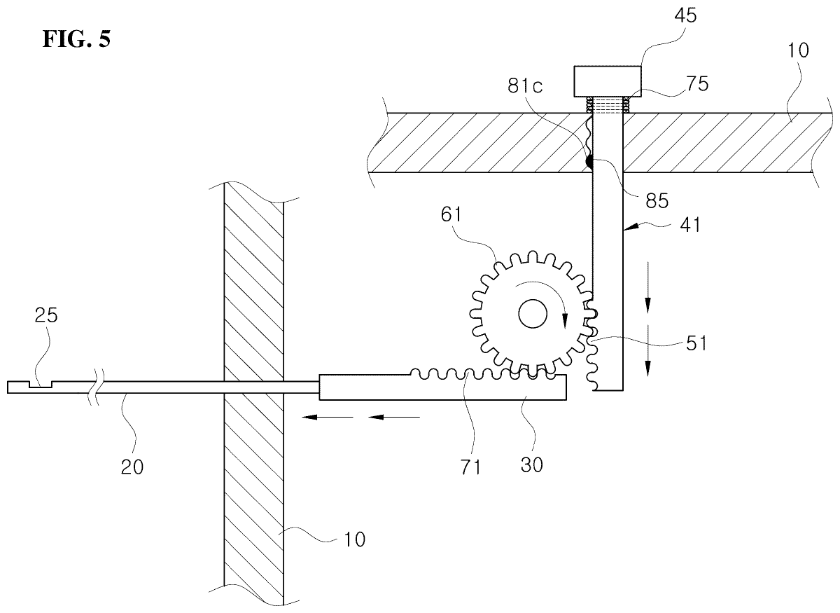

As the elevation rod 41 is secondarily lowered, the driving rack 51 is also secondarily lowered while the elastic member 75 is compressed, and as illustrated in FIG. 5, the driving rack 51 rotates the pinion 61 forward clockwise in correspondence to a distance by which the driving rack 51 is secondarily lowered.

As the pinion 61 rotates forward, the driven rack 71 moves forward toward the procedure area by a secondary movement distance corresponding to a distance by which the driving rack 51 is secondarily lowered in a leftward direction of FIG. 5.

Thus, as the driven rack 71 moves forward by the secondary movement distance, the probe holder 30 and probe 20 also move forward by the secondary movement distance toward the procedure area.

Further, after the probe 20 moves forward by the secondary movement distance toward the procedure area, when the operator releases the push button 45 by further slightly pressing the push button 45 while his/her wrist is fixed so that the locking protrusion 85 is separated from the third locking groove 81c, as illustrated in FIG. 2, by the elastic force generated while the elastic member 75 is tensioned, the elevation rod 41 is raised, the locking protrusion 85 passes through the second locking groove 81b and is locked by the first locking groove 81a, the driving rack 51 is also raised as the elevation rod 41 is raised, and the pinion 61 rotates rearward counterclockwise. Further, as the pinion 61 rotates rearward, the driven rack 71 moves in the rightward direction of FIG. 5 by the movement distance corresponding to the distance by which the driving rack 51 is raised. Thus, the probe 20 and the probe holder 30 move rearward from the procedure area, and as illustrated in FIG. 2, the elevation rod 41 and the probe 20 are restored to their initial positions.

Meanwhile, as an example, when a diabetic retinopathy vitreous body is cut or a diabetic retinopathy removal procedure is performed using the eye-procedure surgical instrument 1 according to the embodiment of the present disclosure, after the push button 45 is pressed so that the locking protrusion 85 is locked by the second locking groove 81b after the first locking groove 81a, when the push button 45 is released so that the locking protrusion 85 is locked by the first locking groove 81a again, the probe 20 may move forward and rearward by the primary movement distance toward the procedure area according to an elevation operation of the elevation rod 41, and the cutting blade 25 provided in the probe 20 may safely and more precisely cut the procedure area while linearly reciprocating on the procedure area without damaging a biological tissue around the procedure area, and thus may shorten a procedure time.

As another example, when a macular procedure or a macular hole procedure is performed using the eye-procedure surgical instrument 1 according to the embodiment of the present disclosure, after the push button 45 is pressed so that the probe 20 moves forward and rearward by the secondary movement distance longer than the primary movement distance toward the procedure area and the locking protrusion 85 is locked by the third locking groove 81c, when the push button 45 is released so that the locking protrusion 85 is locked by the first locking groove 81a again, the probe 20 may move forward and rearward by the secondary movement distance toward the procedure area according to the elevation operation of the elevation rod 41, and the cutting blade 25 provided in the probe 20 may safely and more precisely cut the procedure area while linearly reciprocating on the procedure area without damaging a biological tissue around the procedure area, and thus may shorten a procedure time.

In this way, according to the present disclosure, in an eye procedure requiring a fine procedure of the operator, instead of raising and lowering the entire surgical instrument, the operator may simply operate the surgical instrument using a fine pressing force of the finger in a state in which the wrist is fixed and may finely move the probe having unique procedural functions such as a cutting cutter, forceps, scissors, and a retractor forward and rearward toward the procedure area. Thus, the procedure may be safely and precisely performed without damaging surrounding biological tissues, unskilled people may easily perform the procedure, and the procedure time may also be shortened.

Meanwhile, in the embodiment, the cutting cutter has been described as the probe of the eye-procedure surgical instrument, but the technical spirit according to the present disclosure may be applied to various types of surgical instruments for fine procedures of various biological tissues in addition to the eye.

Hereinabove, the embodiments of the present disclosure have been described with reference to the accompanying drawings. However, those skilled in the art to which the present disclosure pertains may understand that the present disclosure may be implemented in other specific forms without changing the technical spirit or essential features thereof. Therefore, it should be understood that the embodiments described above are illustrative but not limiting in all aspects.

What is claimed is:

1. An eye-procedure surgical instrument comprising:
a handpiece which is grippable;
a probe protruding from the handpiece and configured to perform a procedure of a procedure area of an eye;
a probe holder configured to support one end of the probe and accommodated in the handpiece; and
a probe manipulating unit configured to move the probe forward or rearward with respect to the procedure area,
wherein the probe manipulating unit includes:
an elevation rod elevatably provided in the handpiece to cross a forward/rearward direction of the probe;
a driving rack having a plurality of gear teeth formed at one end of the elevation rod in an elevation direction of the elevation rod;
a pinion having a plurality of gear teeth engaged with the gear teeth of the driving rack and rotated while being engaged with the driving rack according to elevation of the elevation rod; and
a driven rack in which a plurality of gear teeth engaged with the gear teeth of the pinion are formed at one end of the probe holder in the forward/rearward direction of the probe and which moves forward or rearward toward the procedure area by forward/rearward rotation of the pinion.

2. The eye-procedure surgical instrument of claim 1, wherein the probe manipulating unit includes:
a push button provided at a free end of the elevation rod protruding from the handpiece and configured to lower the elevation rod; and
an elastic member provided between the push button and an outer surface of the handpiece and configured to generate an elastic force to raise the elevation rod.

3. The eye-procedure surgical instrument of claim 1, wherein the probe manipulating unit further includes:
a probe movement adjusting unit configured to control lowering of the probe in stages and to finely adjust the forward movement of the probe toward the procedure area.

4. The eye-procedure surgical instrument of claim 3, wherein the probe movement adjusting unit includes:
a plurality of locking grooves recessed in the handpiece at intervals in the elevation direction of the elevation rod; and
a locking protrusion provided on an outer surface of the elevation rod and selectively locked by any one of the plurality of locking grooves.

5. The eye-procedure surgical instrument of claim 1, wherein the probe is any one of a cutting cutter, forceps, scissors, and a retractor.
